(19) United States
(12) Patent Application Publication
(10) Pub. No.: US 2022/0095973 A1
(43) Pub. Date: Mar. 31, 2022

(54) SYSTEMS, METHODS, AND DEVICES FOR MONITORING STRESS ASSOCIATED WITH ELECTRONIC DEVICE USAGE AND PROVIDING INTERVENTIONS

(71) Applicant: METROPOLITAN LIFE INSURANCE CO., New York, NY (US)

(72) Inventor: Terrance Luciani, Monroe Township, NJ (US)

(21) Appl. No.: 17/039,779

(22) Filed: Sep. 30, 2020

(51) Int. Cl. A61B 5/16; A61B 5/11; G16H 10/60; G16H 50/30; G16H 40/67

(52) U.S. Cl. CPC A61B 5/165; A61B 5/1118; G16H 40/67; G16H 50/30; G16H 10/60

(57) ABSTRACT

In one embodiment, a stress monitoring and intervention system receives interaction data from a client device, where the interaction data corresponds to an interaction between a user and one or more client devices. The system also determines the interaction data corresponds to an interaction metric associated with a health profile for the user, and generates a stress value for the interaction metric based on the interaction data. The system further determines the stress value exceeds a stress threshold associated with the interaction metric, selects at least one health intervention, and executes the at least one health intervention to change a user behavior.

### TECHNICAL FIELD

The present disclosure relates generally to smart health systems, and more particularly to a comprehensive system and processes for monitoring stress associated with electronic device usage and providing interventions that drive wellness behaviors.

### BACKGROUND

Electronic devices such as mobile phones, tablets, smart watches, headsets, and so on have become a mainstay in daily life due to increased processing power, decreased costs, improved interfaces, advances in network infrastructure, and the like. The prevalent, convenient, and readily accessible nature of such electronic devices inundates all aspects of work and non-work life. Several studies highlight the potentially addictive and damaging impact caused by constant electronic device usage. Such studies have, in turn, created new opportunities for the consumer electronics industry to measure device usage and provide various charts and graphs representing the same (e.g., screen time graphs, etc.). However, such charts and graphs can be easily ignored and often do not reduce device usage or mitigate stress. In addition, most of the monitoring is localized and only measures usage of a single electronic device, which doesn't account for aggregate stress caused by multiple devices.

The prevalent presence of electronic devices in all aspects of life has also driven growth in the smart health industry. The smart health industry generally attempts to leverage technology to measure a person's health and wellness and provide real-time feedback. While the goals of the smart health industry are to improve a person's health and wellness, most smart health products and solutions generally lead to an increase in device usage in order to gather more data, which often results in increasing the stress caused by constant electronic device usage. The intersection between the goals of the smart health industry and mitigating the damaging effects caused by constant electronic device usage presents new challenges and creates new opportunities to leverage technology and provide meaningful interventions that strike an appropriate balance between device usage and healthy behavior.

### BRIEF DESCRIPTION OF THE DRAWINGS

The embodiments herein may be better understood by referring to the following description in conjunction with the accompanying drawings in which like reference numerals indicate identical or functionally similar elements. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates a schematic diagram of an example smart health environment, showing a stress monitoring and intervention system that communicates with various client devices over a network;

FIG. 2 illustrates a schematic diagram of an example stress monitoring and intervention device;

FIG. 3 illustrates a schematic block diagram of the stress monitoring intervention system shown in FIG. 1, further showing a stress monitoring module/engine, a health profile(s) database, and an intervention module/engine;

FIG. 4A illustrates a table that maps intervention options to respective categories of intervention according to a degree of intervention;

FIG. 4B illustrates a table that maps the categories of intervention to various stress levels;

FIG. 5 illustrates a schematic block diagram of the stress monitoring and intervention system shown in FIG. 3, further showing monitoring and intervention operations;

FIG. 6 illustrates a schematic block diagram of the stress monitoring intervention system shown in FIG. 3, showing exemplary intervention notifications; and

FIG. 7 illustrates an example simplified procedure for stress monitoring and intervention.

### DESCRIPTION OF EXAMPLE EMBODIMENTS

Overview

According to one or more embodiments of the disclosure, a stress monitoring and intervention system receives interaction data from at least one client device, where the interaction data corresponds to an interaction between a user and one or more client devices. The system further determines at least a portion of the interaction data corresponds to an interaction metric associated with a health profile for the user, and generates a stress value for the interaction metric based on the interaction data. The system also determines the stress value exceeds a stress threshold associated with the interaction metric, and selects at least one health intervention from a plurality of health interventions based on the stress value exceeding the stress threshold. The system provides an intervention by executing the at least one health intervention to change a user behavior. These and other features will be discussed herein with respect to various exemplary embodiments of the disclosed stress monitoring and intervention system.

### DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

A smart health environment typically includes a communication network, which is a geographically distributed collection of devices or nodes interconnected by communication links and segments for transporting data there-between. The devices include, for example, electronic devices such as personal computers, laptops, tablets, mobile phones, and the like. Many types of networks are available, ranging from local area networks (LANs) to wide area networks (WANs). LANs typically connect these nodes over dedicated private communications links located in the same general physical location, such as a building or campus. WANs, on the other hand, typically connect geographically dispersed nodes over long-distance communications links, such as common carrier telephone lines, optical lightpaths, synchronous optical networks (SONET), synchronous digital hierarchy (SDH) links, etc.

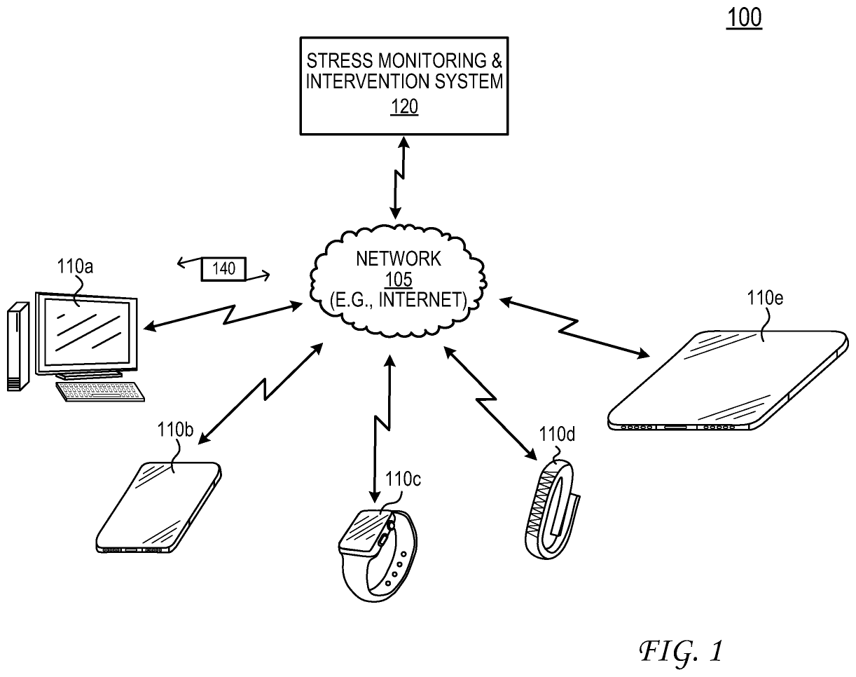

Referring to the figures, FIG. 1 illustrates a schematic diagram of a smart health environment 100, which includes an example communication network 105 (e.g., the Internet). Communication network 105 is shown for purposes of illustration and represents various types of networks, including local area networks (LANs), wide area networks (WANs), telecommunication networks (e.g., 4G, 5G, etc.), and so on.

As shown, communication network 105 includes a geographically distributed collection of devices, such as client devices 110a, 110b, 110c, 110d, and 110e (collectively devices 110). Client devices 110 are interconnected by communication links and/or network segments and exchange or transport data such as data packets 140 to/from a stress monitoring and intervention system 120. As shown, client devices 110 include a computer 110a, a mobile device 110b, a smart watch 110c, a smart wearable device 110d, and a tablet 110e. The illustrated client devices show specific electronic devices, but it is appreciated that client devices 110 in the broader sense are not limited to such specific electronic devices. For example, devices 110 can include any number of electronic devices such as laptops, smart watches, wearable smart devices, smart glasses, vehicle systems, other smart wearables, and so on. In addition, those skilled in the art will understand that any number of devices and links may be used in communication network 105, and that the views shown by FIG. 1 is for simplicity and discussion.

Data packets 140 represent network traffic or messages, which are exchanged between devices 110 and stress monitoring and intervention system 120 over communication network 105 using predefined network communication protocols such as wired protocols, wireless protocols (e.g., IEEE Std. 802.15.4, WiFi, Bluetooth®, etc.), PLC protocols, or other shared-media protocols where appropriate. In this context, a protocol consists of a set of rules defining how devices or nodes interact with each other.

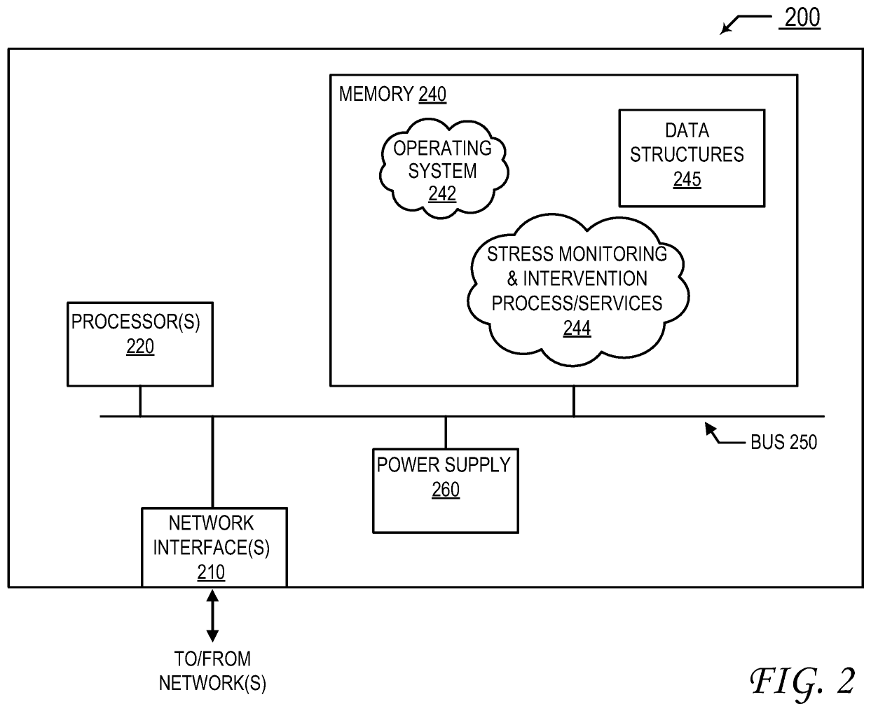

FIG. 2 is a schematic block diagram of an example device 200 that may be used with one or more embodiments described herein, e.g., as a component device of stress monitoring and intervention system 120 and/or as any one of the devices 110 shown in FIG. 1. Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.).

Network interface(s) 210 contain the mechanical, electrical, and signaling circuitry for communicating data over the communication links coupled to communication network 105. Network interfaces 210 may be configured to transmit and/or receive data using a variety of different communication protocols. Network interface 210 is shown for simplicity, and it is appreciated that such interface may represent two different types of network connections, e.g., wireless and wired/physical connections. Also, while network interface 210 is shown separately from power supply 260, for PLC the interface may communicate through power supply 260, or may be an integral component of the power supply. In some specific configurations the PLC signal may be coupled to the power line feeding into the power supply. In addition, it is appreciated that network interfaces 210 can be compatible with the Open Application Program Interface (API) specification to communicate with a variety of new devices, peripherals, etc.

Memory 240 comprises a plurality of storage locations that are addressable by processor 220 and network interfaces 210 for storing software programs and data structures associated with the embodiments described herein. Note that certain devices may have limited memory or no memory (e.g., no memory for storage other than for programs/processes operating on the device and associated caches).

Processor 220 comprises hardware elements or hardware logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may comprise stress intervention process/services 244, described herein. Note that while stress intervention process/services 244 is shown in centralized memory 240, alternative embodiments provide for the process to be specifically operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the stress monitoring and intervention process 244 is shown as a standalone process, those skilled in the art will appreciate that this process may be executed as a routine or module within other processes.

As noted above, a number of studies highlight the addictive and damaging impact constant electronic device usage has on a person. While a number of consumer electronic devices provide various charts and graphs that illustrate device usage, such information is readily and easily ignored. Further, most of the charts and graphs are limited to a single device, which fails to account for an aggregate device usage over multiple devices. At the same time, the smart health industry attempts to improve a person's health and wellness by measuring health metrics, but the products and solutions provided by the smart health industry often leads to an increase in device usage and thus, an increase in the stress caused by constant electronic device usage.

Accordingly, the techniques described herein provide comprehensive smart health monitoring and intervention processes that monitor and quantify stress associated with electronic device(s) usage and provide meaningful interventions that strike an appropriate balance between device usage and healthy behavior. While the techniques discussed herein focus, in part, on quantifying stress associated with electronic device usage, the techniques in the broader sense can quantify general user stress while the user interacts with electronic devices.

These processes are embodied by a smart health monitoring and intervention platform, which includes various systems, devices, and methods discussed herein. At a high level, the platform employs stress monitoring and intervention techniques to provide technical solutions for monitoring interaction data between a user and a user's electronic devices, analyzing the interaction data to quantify a stress (e.g., stress values) associated with electronic device usage, identifying appropriate health interventions (e.g., based on a degree of required intervention), and providing appropriate health intervention(s) to change the user's behavior and mitigate stress.

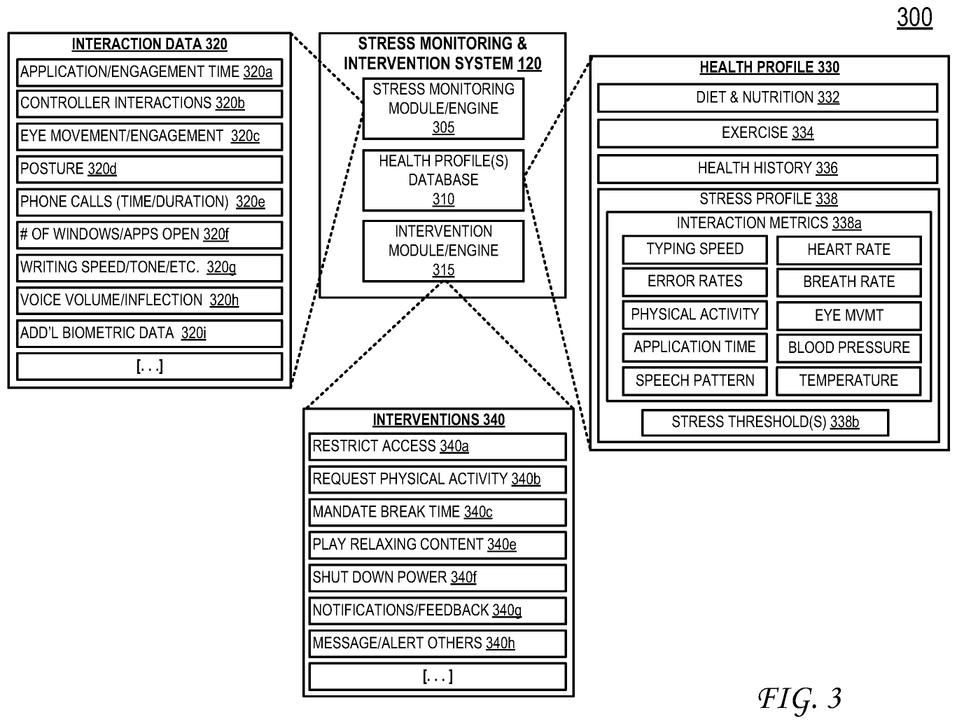

FIG. 3 illustrates a schematic block diagram 300 of stress monitoring and intervention system 120. As shown, stress monitoring and intervention system includes a number of component modules or engines, such as a stress monitoring module 305, a health profile database 310, and an intervention module 315.

In operation, stress monitoring module 305 monitors interaction data 320 from various electronic devices. For example, stress monitoring module 305 monitors interaction data 320 from devices 110 over communication network 105 (ref. FIG. 1). Interaction data 320 corresponds to an interaction between a user and electronic devices (e.g., client devices). Interaction data 320 includes data associated with direct interaction between the user and the electronic device(s) as well as data associated with indirect interaction between the user and the electronic device(s) (e.g., biometric data, etc.).

Here, interaction data 320 includes application or engagement time 320a, controller interactions 320b (e.g., speed, accuracy, error rates, mouse speed, touch-precision, keystrokes, typing speed data, peripheral and/or input data, etc.), eye movement or engagement 320c (e.g., eye tracking), posture 320d (e.g., sitting, standing, etc.), phone calls 320e (e.g., time, duration, tone, etc.), number of windows and/or applications open at a given time 320f, writing speed/tone/etc. 320g (e.g., accuracy, typos, grammatical data, etc.), voice volume/inflection 320h, and other additional biometric data 320i (e.g., heart rate, blood pressure, oxygen level, sleep pattern, and so on.

Collectively, as mentioned, interaction data 320 corresponds to the interaction between the user and the user's electronic devices (e.g., devices 110). In this fashion, interaction data 320 represents data captured during the interaction with the electronic device(s) and/or data that provides context for the interaction between the user and the electronic device(s).

Health profile(s) database 310 represents a repository of health profiles for respective users. Here, health profile(s) database 310 illustrates a health profile 330 for a representative user. Health profile 330 represents a baseline dataset of metrics for the representative user, and includes diet and nutrition metrics 332, exercise metrics 334, health history metrics 336, stress profile metrics 338, and stress thresholds 338b. Health profile 330 is a representative health profile and it may include (or exclude) a number of different factors. For example, health profile 330 can also include information corresponding to location, occupation, marital status, income, and other factors that would impact a person's health baseline.

Stress profile metrics 338 represents a baseline stress dataset for the user associated with health profile 330. Stress profile metrics 338 include a number of interaction metrics 338a, which quantify acceptable baseline levels or metrics of stress associated electronic device usage. Here, interaction metrics 338a include, for example, typing speed, error rates (e.g., typos, grammatical errors, interface errors, touch-errors, interaction-errors, controller-errors, accuracy errors, etc.), application time (e.g., email, Office, social media, etc.), speech pattern (e.g., volume, tone, language, swears, etc.), heart rate, breath rate, eye movement (e.g., eye tracking), blood pressure, temperature, and so on. Collectively, interaction metrics 338a and associated interaction values form a baseline stress profile or stress profile 338.

Stress profile 338 can be established and baselined by monitoring the user associated with health profile 330 for a period of time associated with non-stressful electronic device usage/interaction. Accordingly, stress profile 338 can be specific and unique to an individual. Alternatively, it is also appreciated that larger data sets across similar users, demographics, etc., may be used to establish a universal baseline for the user.

Stress threshold(s) 338b represent boundaries or tolerances. Stress monitoring and intervention system 120 can establish stress threshold(s) 338b based on a variety of factors. For example, stress monitoring and intervention system 120 can evaluate the various datasets within health profile 330 (e.g., diet and nutrition 332, exercise 334, and stress profile 338) to create appropriate stress threshold(s) 338b for the user. In this fashion, an individual that has a healthy diet, maintains a regular exercise regime, and has a clean health history can have higher stress threshold(s) 338b as compared to an individual that does not.

It is appreciated that stress threshold(s) 338b can include specific tolerances or thresholds for each metric of interaction metric 338a. Alternatively, it is appreciated that stress monitoring and intervention system 120 may quantify, weight, and scale interaction metrics 338a to generate a single baseline stress score and determine a single corresponding stress threshold 338b. In some examples, a user can adjust stress threshold(s) 338b and/or a user can add additional complimentary thresholds.

In operation, stress monitoring and intervention system 120 monitors interaction data 320 and based on the interaction data, it further determines stress values corresponding to interaction metrics 338a. Stress monitoring and intervention system 120 also compares the stress values for interaction metrics 338a to one or more stress threshold(s) 338b to determine when intervention 340 is required and executes the appropriate intervention. As mentioned, in some examples, stress monitoring and intervention system 120 computes a single stress value, compares that single stress value to the baseline stress score, and executes an intervention when the single stress value exceeds the baseline score by a stress threshold.

Interventions 340 represent a variety of tools or processes to remediate and mitigate the impact of stress caused by device usage and change a user behavior. Interventions 340 includes remedial actions such as restrict access 340a, request (or require) physical activity 340b, mandate break time 340c, play relaxing content 340e, shut down power 340f, provide notifications or feedback 340g (e.g., including a quiz to further assess the user's stress level), provide messages and/or alerts to others 340h, and so on.

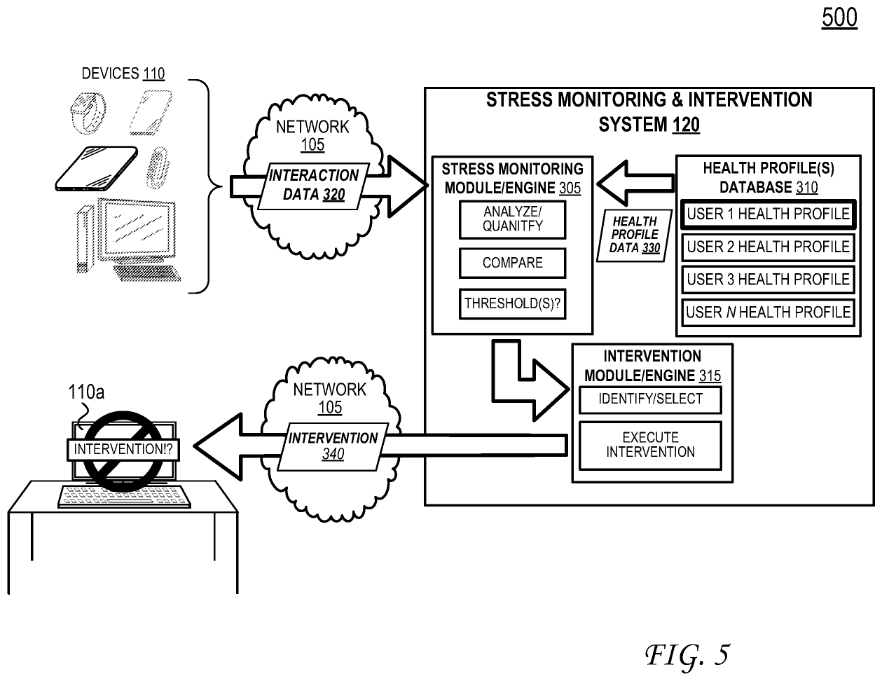

Collectively, stress monitoring and intervention system 120 monitors, processes, analyzes, and quantifies interaction data 320 to determine stress values for interaction metrics such as interaction metrics 338a. Stress monitoring and intervention system 120 can monitor interaction data 320 from one device as well as from an aggregate number of devices. For example, interaction data 320 can monitor eye movement 320c from a mobile device as well as posture 320d from another wearable smart health device to capture the comprehensive stress caused by electronic device usage. Stress monitoring and intervention system 120 further determines when the quantified stress for the user exceeds one or more stress threshold(s) 338b, which triggers an intervention 340. These and other processes are further discussed with reference to FIG. 5 (below).

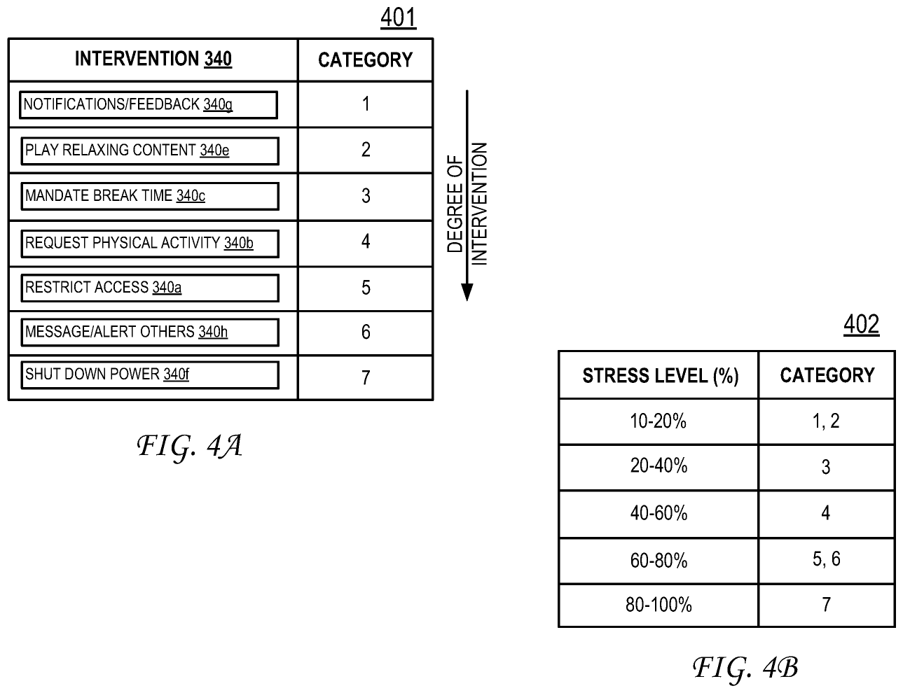

FIGS. 4A and 4B illustrate respective tables 401 and 402 that organize interventions 340 into respective categories (table 401), and map the categories to respective stress levels or thresholds (table 402).

In particular, table 401 of FIG. 4A illustrates a mapping between each intervention 340 to a respective category based on a degree of intervention. Here, a greater, increased, and/or higher category number is associated with a more invasive, greater, and/or increased degree of intervention. For example, shutting down power 340f is assigned to category 7, which is considered to be a greater or more invasive intervention than notifications/feedback 340g (category 1). In other words, the degree of intervention represents a degree of impact on the interactions between the user and the device. The more invasive or greater degree of intervention corresponds to an increased restriction on the interaction between the user and the device(s). The greatest degree of intervention shown in table 401 is category 7, which ceases or stops the interaction between the user and the device.

It is also appreciated that multiple interventions can overlap and/or be assigned to the same category, and it is also appreciated that the same intervention may have a different period of time for a different stress level. For example, restricting access 340a is assigned to category 5, which can correspond to restricting access to the device(s) for 10 minutes. Restricting access 340a can also be assigned to a lower (or higher category) based on more or less restriction time. In this fashion, the interventions are provided for representation and illustration purposes—any number of varying types of interventions may be used and the interventions shown may include a number of sub-interventions to capture different degrees of intervention nuance.

Table 402 shown in FIG. 4B assigns each category to a stress level or stress range, shown as a percentage. The stress levels or stress ranges correspond to stress threshold(s) 338b and represent an amount of stress over a baseline (e.g., 0-10%). The stress levels are shown as percentages for simplicity and discussion, and it is appreciated that any quantifiable range may be used—e.g., 0-10, 0-100, etc. In operation, stress monitoring and intervention system 120 selects an appropriate intervention based on the assigned category and stress level. As discussed above, it is appreciated that the stress levels can be modified and assigned to specific interaction metrics 338a, and/or the interaction metrics can be combined, weighted, and scaled into a single comprehensive stress score. In general, an increased or greater stress level maps to a category of increased or greater degree of intervention.

Collectively, tables 401 and 402 represent one approach to assess and score a risk/stress level and select appropriate intervention(s). The interventions can include, for example, flashing a warning that the current work level is harmful (notifications/feedback 340g), prompting a user quiz to determine whether a break is warranted (notifications/feedback 340g), and requiring a break for a period of time (mandate break time 340c). Further, for critically high risk or stress levels score, the interventions could include automatic rescheduling calendar events (message/alert others 340h), or even shutting down the device(s) (restrict access 340a/shut down power 340f). It is appreciated that all interventions can be controlled and overruled by the user and/or system administrator. For example, it is appreciated that the user may have an urgent deadline, which requires non-stop work for a long period of time. The user and/or administrator can set exceptions to increase the stress levels or withhold interventions altogether. It is also appreciated that intervention 340 can represent and conform to requirements based on union contracts, OSHA and/or government regulations, and compliance with other laws.

FIG. 5 illustrates a schematic block diagram 500 of the stress monitoring and intervention system 120, showing the stress monitoring and intervention operations. In operation, stress monitoring and intervention system 120 continuously monitors devices 110 (associated with a user) over network 105 to obtain interaction data 320. Stress monitoring and intervention system 120 further analyzes or quantifies interaction data 320 to determine stress values for corresponding interaction metrics (e.g., interaction metrics 338a). Stress monitoring and intervention system 120 retrieves and/or looks up the interaction metrics associated with a user's health profile—User 1 Health Profile—and compares the stress values to the interaction metrics therein. Stress monitoring and intervention system 120 further determines when the stress values for the corresponding interaction metric exceed a threshold, which triggers an intervention. Stress monitoring and intervention system 120 identifies or selects the appropriate intervention based on the level of stress, and executes (e.g., pushes) the intervention to corresponding electronic devices—e.g., client device 110a—to change a user behavior and mitigate or reduce the stress associated with device usage.

It is also appreciated that stress monitoring and intervention system 120 actively and iteratively updates health profile 330 for a user. For example, stress monitoring module 305 monitors devices to determine and establish baseline health metrics stored in the user's health profile 330 and continues to periodically update the baseline health metrics. In this fashion, health profile 330 reflects a significant body of health history information about a given user, the user's stress, and broad picture of the user's overall health.

As illustrated, stress monitoring module 305 monitors, analyzes, quantifies, compares, and determines thresholds; health profile(s) database 310 stores and provides the appropriate health profile (User 1 Health Profile/health profile data 330) with corresponding interaction metrics for the user; and intervention module 315 performs the intervention, which includes identifying, selecting, and executing the intervention on client device 110a.

In some embodiments, stress monitoring and intervention system 120 includes a distributed software application that executes on all devices 110. In other embodiments, stress monitoring and intervention system 120 includes a consolidated software application that communicates with devices 110 through various known protocols (e.g., Application Program Interfaces (APIs), etc.).

In addition, stress monitoring and intervention system 120 may comprise an enterprise program that evaluates user stress on a macro level such as work groups, divisions, etc. In such examples, the system could detect issues within specific departments or specific groups of users that are all assigned to a stressful project, and mitigate the stress before it impacts their work and/or causes accidents (e.g., Air Traffic Controllers, heavy machine operators, etc.)

Stress monitoring and intervention system 120 continuously measures interaction data 320 associated with a user and quantifies interaction data 320 into stress values to understand the user's performance and stress. The stress values can represent for example, a length of time that someone is working, how they are working, whether they have taken the appropriate breaks, and a level of distress impacting the body. Stress monitoring and intervention system 120 compares the stress values to corresponding interaction metrics and thresholds to determine critical levels of stress. Stress monitoring and intervention system 120 iteratively monitors interaction data 320 and continues to refine and update its stress assessment.

For example, stress monitoring and intervention system 120 can monitor a smartwatch for biometric data such as heart rate, blood pressure, and O2 level. Stress monitoring and intervention system 120 can also monitor mobile devices to determine a lack of physical activity or movement (e.g., indicated by GPS data, accelerometer data, etc.). Stress monitoring and intervention system 120 can also monitor application information such as calendar data, emails sent, and the activity level of a user's computer, which may indicate a significant amount of time working. Stress monitoring and intervention system 120 can also monitor peripheral interactions such as keystrokes and grammatical mistakes. Stress monitoring and intervention system 120 can also determine that too many windows are open and/or too many programs are running, which indicates a level of distractedness (or stress). Stress monitoring and intervention system 120 can also identify phone usage, the number of phone calls in a set period of time, voice volume and tone, and stress implications of certain phone numbers. Stress monitoring and intervention system 120 can leverage built in device cameras to detect attentiveness, posture, movement/motion (e.g., sitting a desk daydreaming), and so on. Stress monitoring and intervention system 120 can also monitor application specific data to detect tone in email.

The comprehensive nature of monitoring interaction data 320 aggregates data over multiples devices and multiple interaction metrics and allows stress monitoring and intervention system 120 to separate eustress from distress and identify specific periods of potentially harmful impact.

For the example cases below, references are made to various aspects of stress monitoring and intervention system 120 shown in FIGS. 3, 4A, 4B, and 5.

Example Case #1—Warning Sign Intervention.

In this example, the user has been working for four hours straight with continuous typing and activity. Posture is poor and the user has not moved. There has been no other activity but typing.

Stress monitoring and intervention system 120 monitors interaction data corresponding to the following datasets: health information including heart rate and breathing (additional biometric data 320i); camera information including blink rate and posture (eye movement/engagement 320c and posture 320d); activity information including movement (additional biometric data 320i), typing speed (writing speed/tone/etc. 320g); and cognitive information including words typed, system use, and voice signals (# of windows/apps open 320f, voice volume/inflection 320h, and additional biometric data 320i). Stress monitoring and intervention system 120 could also evaluate a number of additional datasets (not shown) such as diet and nutrition information (e.g., when the user last ate), exercise regimes (e.g., when did the user last workout), and so on. All of these datasets build context for the current interaction between the user and the user's devices, and help the system identify stressful conditions.

Stress monitoring and intervention system 120 receives the interaction data corresponding to the foregoing datasets and quantifies the interaction data into stress values for corresponding interaction metrics associated with the user's health profile 330/stress profile 338. For example, stress monitoring and intervention system 120 quantifies the interaction data into stress values for interaction metrics 338a such as typing speed, error rates, eye movement, physical activity, breath rate, and heart rate. As mentioned, in some examples, stress monitoring and intervention system 120 generates a single baseline stress score and a single threshold 338b for interaction metrics 338a. In these examples, stress monitoring and intervention system 120 quantifies interaction data 320 into a single stress value and compares the stress value to the baseline stress score to determine if the stress value exceeds the threshold.

In this case, stress monitoring and intervention system 120 determines the stress values exceed the corresponding stress threshold(s) 338b, resulting in a 10-20% stress level above baseline. Stress monitoring and intervention system 120 identifies appropriate intervention category(ies) assigned to stress level 10-20% (table 402). Here, stress monitoring and intervention system 120 identifies interventions corresponding to categories 1 and 2 from table 402, which include notifications/feedback 340g and play relaxing content 340e. The interventions corresponding to play relaxing content 340e and notifications/feedback 340g include, for example, playing classical music, providing a popup notification that requests the user to stand, executing an interactive program to perform a breathing exercise, providing a popup quiz to further evaluate the stress level, and so on.

Example #2—Potential Harm Intervention.

In this example, the user has had a difficult phone call after 4 continuous 1-hour meetings. During the calls, the user's voice pattern is faster than normal and louder than normal. The user's emails during this time include several grammatical errors and a stressful tone.

Stress monitoring and intervention system 120 monitors interaction data corresponding to the following datasets: cognitive information corresponding to phone usage and voice signals (application/engagement time 320a, voice volume/inflection 320h), email information (writing speed/tone/etc. 320g), health information (additional biometric data 320i), and camera information including blink rate and posture (eye movement/engagement 320c and posture 320d).

Stress monitoring and intervention system 120 receives the interaction data corresponding to the foregoing datasets and quantifies the interaction data into stress values for corresponding interaction metrics associated with the user's health profile 330/stress profile 338. For example, stress monitoring and intervention system 120 quantifies the interaction data into stress values for interaction metrics 338a such as typing speed, error rates, application time, physical activity, heart rate, and speech pattern.

Stress monitoring and intervention system 120 further determines the stress values exceed the corresponding stress threshold(s) 338b, resulting in a 60-80% stress level above baseline. Stress monitoring and intervention system 120 identifies appropriate interventions category(ies) assigned to stress level 60-80% as well as the interventions assigned to the levels leading up to 60-80% (table 402) to mitigate the stress. In other words, stress monitoring and intervention system 120 escalates the degree of intervention as the stress level rises.

Here, stress monitoring and intervention system 120 employs interventions corresponding to categories 1 and 2 for stress levels 10-20%, interventions corresponding to category 3 for stress levels 20-40%, interventions corresponding to categories 3 and 4 for stress levels 40-60%, and interventions corresponding to categories 4, 5, and 6 for stress levels 60-80%.

In this case, stress monitoring and intervention system 120 employs similar interventions to those discussed in Example #1 for stress levels 10-20%—e.g., notifications/feedback 340g and playing relaxing content 340e. As stress monitoring and intervention system 120 continues to monitor the interaction data, it determines the stress levels reach 20-40% and employs interventions corresponding to category 3—e.g., mandate break time 340c. The interventions for mandate break time 340c can include a required break for a period of time such as 10 minutes. At 40-60% stress level, stress monitoring and intervention system 120 requests a physical activity 340b intervention, which can require the user to take a walk. The physical activity 340b intervention can be verified by accelerometer data, GPS data, etc. when the user move the electronic device (e.g., mobile phone, smart health wearable device, etc.) during the walk. At 60-80% stress monitoring and intervention system 120 employs interventions corresponding to category 5 to restrict access 340a to one or more devices and category 6 to message/alert others 340h. The interventions represented by restrict access 340a can include imposing delays for certain applications (e.g., a 5 minute delay before sending an email), imposing a time limit for accessing applications on the device, locking the device, automatically scheduling and calendaring a break in the user's calendar, requiring approval from a manager or other party to grant access again, and so on. The interventions represented by message/alert others 340h can include sending an alert to another party (e.g., a manger, HR, etc.), requiring the other party's approval before the user performs additional work (e.g., reviewing/approving an email before the user can send it), enrollment into an employer-sponsored stress management class, and so on.

Example Case #3—Critical Intervention.

In this example, the user has been under a significant or critical amount of stress due, in part, to contestant device usage over an extended period. The user's behavior indicates the user is dealing with a personal crisis, which is impacting their presentation. For example, the user may have sent inappropriate emails, the user may be speaking with a raised voice, and/or the work activity or work performance may have significantly deviated from the average level.

Stress monitoring and intervention system 120 monitors interaction data corresponding to the following datasets: health information including heart rate and blood pressure (additional biometric data 320i); camera information including eye movement and general engagement (eye movement/engagement 320c); application information (application time 320); and cognitive information including words typed, errors, tone (writing speed/tone/etc. 320g).

Similar to the above example cases, stress monitoring and intervention system 120 receives the interaction data corresponding to the foregoing datasets and quantifies the interaction data into stress values for corresponding interaction metrics associated with the user's health profile 330/stress profile 338. Here, stress monitoring and intervention system 120 quantifies the interaction data into stress values for interaction metrics 338a such as typing speed, error rates, eye movement, blood pressure, and heart rate.

Stress monitoring and intervention system 120 further compares the stress values against the baseline interaction metrics to determine the stress values exceed the corresponding stress threshold(s) 338b, which results in a critical 80-100% stress level above baseline, or a stress crisis. Stress monitoring and intervention system 120 identifies appropriate intervention category(ies) assigned to each stress level shown in table 402, and as mentioned, stress monitoring and intervention system 120 employs the corresponding intervention assigned to the category(ies) for each stress level. In addition to the interventions corresponding to categories 1-6, stress monitoring and intervention system 120 employs category 7 interventions, which includes shutting down power 340f (e.g., shutting down the device for 24 hours). Additional category 7 interventions can include sending messages/alerts to managers and Human Resources (HR) that detail the impact and severity of the crisis as well as automatically scheduling a meeting with an Employer Assistance Program.

Importantly, the interventions for each case scenario can vary based on a set of pre-programmed response actions, and the interventions can be approved by appropriate legal/compliance teams to protect privacy (e.g., HIPPA law compliance) and adhere to all relevant customs, regulations, contracts, and laws.

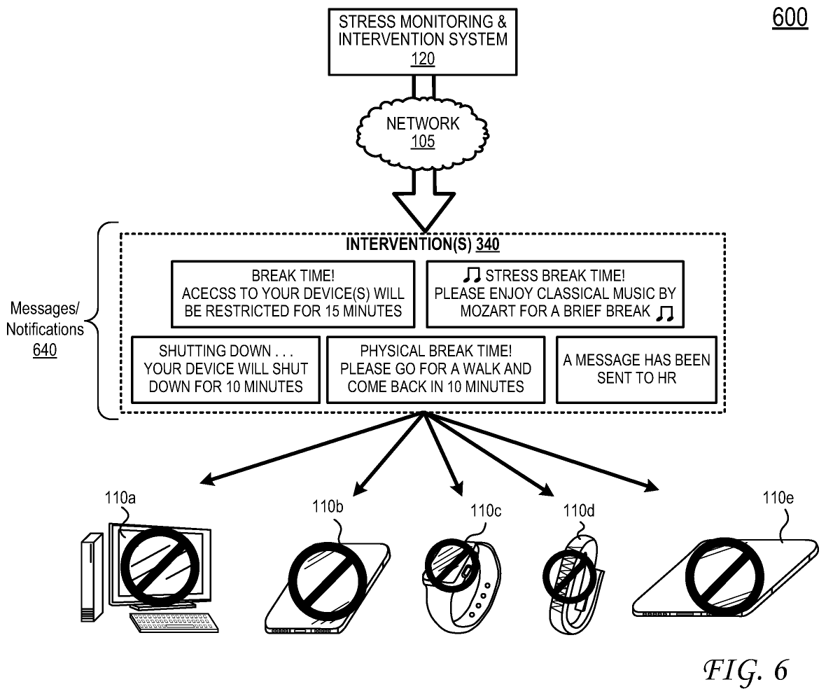

FIG. 6 illustrates a schematic block diagram 600 of stress monitoring intervention system 120, further showing exemplary interventions 340 in the form of notifications or messages 640. As discussed, stress monitoring and intervention system 120 monitors the interaction data from an aggregate number of electronic devices (e.g., client devices 110a-e), determines when the stress values exceed a threshold, and executes interventions 340 by pushing or transmitting intervention data to one or more client devices. Depending on the configuration, stress monitoring and intervention system 120 sends or pushes interventions to the appropriate client device(s) to maximize stress reduction. In other words, stress monitoring and intervention system 120 can send an intervention to a single client device or multiple client devices depending on the stress level. In FIG. 6, stress monitoring and intervention system 120 sends messages 640 to all of client devices 110a-e.

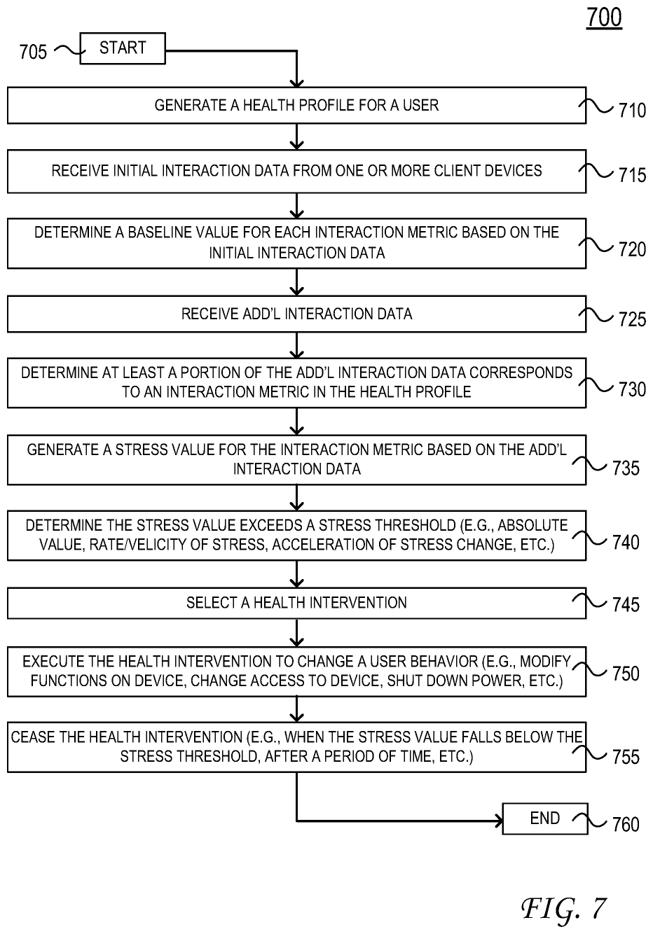

FIG. 7 illustrates an example simplified procedure 700 for stress monitoring and intervention in accordance with the processes described above. For purposes of discussion, the operations for procedure 700 are described in the context of the stress monitoring and intervention system 120 or simply the “system.”

Procedure 700 begins at step 705, and continues to step 710, where, as described in greater detail above, the system generates a health profile for a user. The health profile includes health and wellness information about the user such as the information found in health profile 330 (ref. FIG. 3). Notably, the health profile includes a stress profile having a plurality of interaction metrics (e.g., FIG. 3; interaction metrics 338a) and stress threshold(s) 338b). In some examples, the user can access and modify his/her health profile and/or other applications can request access to the health profile to provide, update, or otherwise modify appropriate health profile baseline metrics. In addition, the health profile can store a historical record of “stress” or “stress” history for the user. The cumulative impact of such stress over time may further impact the stress thresholds for that given user.

Procedure continues to step 715, where the system monitors and receives initial interaction data from one or more client devices. In some examples, the system determines the user initiated an interaction with a first client device, such as a mobile phone. In these examples, the system can request interaction data such as biometric data from a second client device, such as a wearable health device based on the user-initiated interaction. In this fashion, the system gathers various types of interaction data from multiple devices to create a comprehensive context for evaluating stress.

Next, in step 720, the system determines baseline values for each interaction metric based on the initial interaction data to establish a baseline stress profile. The baseline values represent non-stressed user behavior or metrics. It is appreciated that system can determine the baseline values for a specific user in a controlled non-stressed environment, and/or the system can determine baseline values for a number of users and establish average baseline values for similarly situated users (e.g., based on demographic information, work environment, device usage, etc.). For example, a heart surgeon or firefighter can have different baseline stress profiles than clerical assistants.

The system receives additional interaction data at step 725. As mentioned, the interaction and/or the additional interaction data corresponds to an interaction or activity between the user and one or more client devices. Accordingly, the interaction data represents data captured during an interaction with the client device(s) and/or data that provides context for the interaction between the user and the client device(s).

At step 730, the system determines at least a portion of the additional interaction data corresponds to an interaction metric in the health profile associated with the user. As one example, the system can determine that writing speed data (e.g., FIG. 3; writing speed/tone/etc. 320g) corresponds to a typing speed interaction metric (e.g., FIG. 3; interaction metrics 338a—typing speed).

The system also generates a stress value for the interaction metric based on the additional interaction data at step 735. Next, at step 740, the system determines the stress value exceeds a stress threshold associated with the interaction metric. Continuing with the example above, the system can determine a stress value corresponding to the words-per-minute typed by the user. In this example, the system compares the words-per-minute to the baseline value for the corresponding interaction metric—e.g., the baseline words-per-minute for “typing speed.” The system further determines when the stress value exceeds or deviates from the baseline value for the corresponding interaction metric. For example, if the words-per-minute value is below (or above) a threshold number relative to the baseline value, the system determines a stress condition exists. Notably, the stress threshold can represent an absolute value, a rate of a stress change in a given time period, and/or an acceleration of a stress change in the given time period.

Once the stress threshold is exceeded, the system further selects a health intervention at step 745. As discussed, the system can assign or categorize each health intervention into one or more stress levels based on a degree of intervention (ref. FIG. 4A; table 401). The system can further assign the categories to corresponding stress levels (ref. FIG. 4B; table 402). The system can determine a stress level based on the comparison between the stress value, the baseline value, and the stress threshold, and select the health intervention at step 745 based on a stress level. Again, while the stress levels discussed and illustrated by this disclosure include percentage ranges, it is appreciated that any stress level scale may be used—e.g., 0-10, 0-100, etc. It is also appreciated that while step 745 indicates the system selects one health intervention, it is appreciated that the system can select any number of health interventions corresponding to the stress level, as well as health interventions leading up to the stress level.

At step 750, the system executes the health intervention to change a user behavior, which interventions can include, modifying functions on device, change access to device, modifying access to applications, sending messages or alerts, changing calendared events or tasks, shutting down power, and so on.

In some examples, the system can run “stress tests” similar to fire drills. In these examples, the system can shutdown all activity and access to electronic devices to better teach people how to manage stress in high pressure situations. If the stress detected during these tests exceeds a benchmark, the system records the failure to maintain an adequate stress level and shuts down. Continued use can help the end user become better at managing high stress situations.

The system continues to monitor the interaction data and can cease, at step 755, the health intervention when the stress value falls below the stress threshold and/or after a set period of time passes. In this fashion, the system continuously updates the stress value based on subsequent interaction data, compares the stress value to the baseline value and/or stress threshold associated with the interaction metric, and stops or removes the health intervention when the stress value no longer exceeds the stress threshold.

Procedure 700 subsequently ends at step 760, but may continue on to step 710 where, as discussed above, the system generates a health profile for the user. It should be noted that certain steps within procedure 700 may be optional, and further, the steps shown in FIG. 7 are merely examples for illustration—certain other steps may be included or excluded as desired. Further, while a particular order of the steps is shown, this ordering is merely illustrative and any suitable arrangement of the steps may be utilized without departing from the scope of the embodiments herein.

The techniques described herein, therefore, describe comprehensive stress monitoring and intervention processes that evaluate interaction data between a user and multiple electronic devices. The techniques evaluate interaction data to determine stress associated with electronic device usage as well as the broader underlying contextual stress associated with user while using the electronic device. The techniques quantify the interaction data to determine a stress level of the user and provide an appropriate intervention to mitigate or reduce the stress and ultimately, protect the user.

While there have been shown and described illustrative embodiments of a stress monitoring and intervention system, it is to be understood that various other adaptations and modifications may be made within the spirit and scope of the embodiments herein. For example, the embodiments have been shown and described herein with relation to a specific system that organizes certain functions into modules or engines. However, the embodiments in their broader sense are not as limited, and may, in fact, be used with any number of applications, devices, and systems as part of a distributed computing network.

The foregoing description has been directed to specific embodiments. It will be apparent, however, that other variations and modifications may be made to the described embodiments, with the attainment of some or all of their advantages. For instance, it is expressly contemplated that the components and/or elements described herein can be implemented as software being stored on a tangible (non-transitory) computer-readable medium, devices, and memories (e.g., disks/CDs/RAM/EEPROM/etc.) having program instructions executing on a computer, hardware, firmware, or a combination thereof. Further, methods describing the various functions and techniques described herein can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on. In addition, devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example. Instructions, media for conveying such instructions, computing resources for executing them, and other structures for supporting such computing resources are means for providing the functions described in these disclosures. Accordingly, this description is to be taken only by way of example and not to otherwise limit the scope of the embodiments herein. Therefore, it is the object of the appended claims to cover all such variations and modifications as come within the true spirit and scope of the embodiments herein.

1. A monitoring and intervention system, comprising:
one or more network interfaces configured to communicate with a plurality of client devices over communication network;
a processor coupled to the network interfaces and adapted to execute one or more processes; and
a memory configured to store instructions executable by the processor, the instructions, when executed, are operable to:
receive interaction data from at least one client device, the interaction data corresponding to an interaction between a user and one or more client devices;
determine at least a portion of the interaction data corresponds to an interaction metric associated with a health profile for the user;
generate a stress value for the interaction metric based on the interaction data;
determine the stress value exceeds a stress threshold associated with the interaction metric;
select at least one health intervention from a plurality of health interventions based on the stress value exceeding the stress threshold; and
execute the at least one health intervention to change a user behavior.

2. The monitoring and intervention system of claim 1, wherein the instructions are further operable to:
generate the health profile for the user, the health profile including a baseline stress profile having a plurality of interaction metrics;
receive initial interaction data from one or more of the plurality of client devices; and
determine a baseline value for each interaction metric of the plurality of interaction metrics based on the initial interaction data.

3. The monitoring and intervention system of claim 1, wherein the instructions are further operable to:
monitor one or more of the plurality of client devices for subsequent interaction data;
update the stress value based on the subsequent interaction data;
determine the stress value is below the stress threshold associated with the interaction metric; and
cease the at least one health intervention based on the stress value being below the stress threshold.

4. The monitoring and intervention system of claim 1, wherein the instructions, when executed, are further operable to:
cease the at least one health intervention after a predetermined period of time.

5. The stress monitoring and intervention system of claim 1, wherein the instructions to execute the at least one health intervention are further operable to:
modify one or more functions associated with one or more client devices associated with the user.

6. The monitoring and intervention system of claim 1, wherein the instructions are further operable to:
assign each health intervention of the plurality of health interventions to one or more stress levels based on a degree of intervention;
determine the stress value corresponds to a first stress level of the one or more stress levels; and
wherein the instructions to select the at least one health intervention are further operable to select the at least one health intervention assigned to the first stress level.

7. The monitoring and intervention system of claim 6, wherein an increased stress level corresponds to an increased degree of intervention.

8. The monitoring and intervention system of claim 1, wherein the interaction data includes biometric data, wherein the instructions to receive interaction data from the at least one client device are further operable to:
determine the user initiated an interaction with a first client device of the plurality of client devices; and
request the biometric data from a second client device of the plurality of client devices based on the interaction with the first client device.

9. The monitoring and intervention system of claim 1,
wherein the instructions to determine the stress value exceeds the stress threshold are further operable to determine the stress value exceeds the stress threshold for a predetermined number of times in a time period, and
wherein the instructions to select the at least one health intervention are further operable to select the at least one health intervention based on a rate of change of the stress value.

10. The monitoring and intervention system of claim 1, wherein the interaction data includes at least one of biometric data, typing speed data, input data, grammatical data, interaction accuracy data, application time data, or speech pattern data.

11. The monitoring and intervention system of claim 1, wherein the at least one health intervention includes at least one of playing content by a client device, sending a notification, sending a message to another user, requesting activity by the user, restricting access between the user and one or more programs on the client device, restricting access between the user and the client device, or shutting down power to the client device.

12. A method, comprising:
receiving, by a processor, interaction data from at least one client device over a communication network, the interaction data corresponding to an interaction between a user and one or more of a plurality of client devices;
determining, by the processor, at least a portion of the interaction data corresponds to an interaction metric associated with a health profile for the user;
generating a stress value for the interaction metric based on the interaction data;
determining the stress value exceeds a stress threshold associated with the interaction metric;
selecting at least one health intervention from a plurality of health interventions based on the stress value exceeding the stress threshold; and
executing the at least one health intervention to change a user behavior.

13. The method of claim 12, further comprising:
generating the health profile for the user, the health profile including a baseline stress profile having a plurality of interaction metrics;
receiving initial interaction data from one or more of the plurality of client devices; and
determining a baseline value for each interaction metric of the plurality of interaction metrics based on the initial interaction data.

14. The method of claim 12, further comprising:
monitoring one or more of the plurality of client devices for subsequent interaction data;
determining the stress value is below the stress threshold associated with the interaction metric; and
ceasing the at least one health intervention based on the stress value being below the stress threshold.

15. The method of claim 12, further comprising:
ceasing the at least one health intervention after a predetermined period of time.

16. The method of claim 12, wherein executing the at least one health intervention further comprises:
modifying one or more functions associated with one or more client devices associated with the user.

17. The method of claim 12, wherein the interaction data includes biometric data, wherein receiving the interaction data from the at least one client device further comprises:
determining the user initiated an interaction with a first client device of a plurality of client devices; and
requesting the biometric data from a second client device of the plurality of client devices based on the interaction with the first client device.

18. A tangible, non-transitory, computer-readable media having instructions encoded thereon, the instructions, when executed by a processor, are operable to:
receive interaction data from at least one client device, the interaction data corresponding to an interaction between a user and one or more of a plurality of client devices;
determine at least a portion of the interaction data corresponds to an interaction metric associated with a health profile for the user;
generate a stress value for the interaction metric based on the interaction data;
determine the stress value exceeds a stress threshold associated with the interaction metric;
select at least one health intervention from a plurality of health interventions based on the stress value exceeding the stress threshold; and
execute the at least one health intervention to change a user behavior.

19. The tangible, non-transitory, computer-readable media of claim 18, wherein the instructions, when executed by a processor, are further operable to:
generate the health profile for the user, the health profile including a baseline stress profile having a plurality of interaction metrics;
receive initial interaction data from one or more of the plurality of client devices; and
determine a baseline value for each interaction metric of the plurality of interaction metrics based on the initial interaction data.

20. The tangible, non-transitory, computer-readable media of claim 18, wherein the instructions, when executed by a processor, are further operable to:
monitor one or more of the plurality of client devices for subsequent interaction data;
update the stress value based on the subsequent interaction data;
determine the stress value is below the stress threshold associated with the interaction metric; and
cease the at least one health intervention based on the stress value being below the stress threshold.
